(19) United States
(12) Patent Application Publication
(10) Pub. No.: US 2017/0211047 A1
(43) Pub. Date: Jul. 27, 2017

(54) METHOD FOR CELL REPROGRAMMING AND DIFFERENTIATION BY MICROFLUIDIC TECHNOLOGY

(71) Applicant: USTEM S.R.L, PADOVA (IT)

(72) Inventors: Nicola Elvassore, Padova (IT); Camilla Luni, Padova (IT); Stefano Giulitti, Istrana (Treviso) (IT)

(21) Appl. No.: 15/006,505

(22) Filed: Jan. 26, 2016

(51) Int. Cl. C12N 5/074

(52) U.S. Cl. CPC C12N 5/0696; C12N 2500/02; C12N 2506/02

(57) ABSTRACT

A method for reprogramming differentiated cells and for then converting the reprogrammed cells into a differentiated phenotype of interest by means of microfluidic technology is described, together with a related kit for cell reprogramming.

### CROSS REFERENCE TO RELATED APPLICATIONS

The present application is related to Italian Patent Application PD2013A000220 filed on Aug. 2, 2013 and made available to the public on Feb. 2, 2015, which application is incorporated herein by reference in its entirety.

### TECHNICAL FIELD

The present disclosure provides a methodology for reprogramming differentiated cells and subsequently differentiating them, in both cases at the microscale level.

### BACKGROUND

Methods for cell reprogramming and differentiation are known.

Specifically, the term “reprogramming” indicates the process that is used to de-differentiate differentiated cells to cells that are immature and pluripotent, also known as induced pluripotent stem cells (iPSc), and the term “differentiation” indicates the process used to differentiate immature and pluripotent cells into cells derived from the three germ layers.

Currently, reprogramming is performed in laboratories within standard devices for cell culture, like Petri dishes and multi-well plates, which however show multiple limitations.

First, to fully take advantage of the potential of reprogramming for scientific and therapeutic purposes, the process should be applied to human somatic cells derived from a healthy or diseased patient. Thus, it is necessary to perform reprogramming from a small number of cells.

Second, due to the high variability between biological samples, it is necessary to reprogram a high number of samples to obtain statistically significant data. This implies using a large amount of chemical and biological reagents and high costs of manual labor, making the process economically unsustainable at large scale. Moreover, current methodologies to produce iPSc and to possibly subsequently produce differentiated cells after iPSc expansion require time-consuming procedures that last weeks and are not sustainable.

The term “expansion” indicates the stage when cells that are part of an iPSc colony are propagated: typically, iPSc colonies are manually dissected and the cells in the colony fragments are seeded in other dishes, where by cell division produce new colonies, etc.

Thus, there is a strong need for new methodologies and new techniques to perform cell reprogramming and the possible subsequent cell differentiation in a consistent, rapid and affordable manner.

Microfluidics is a recent multidisciplinary science that deals with small liquid volumes, from microliters down to nanoliters, within devices having at least one micrometric dimension.

From a practical standpoint, at nanometer scale fluids can demonstrate a very different behavior compared to that at macroscale, and this should be kept in mind when microfluidic devices are designed or experiments within these devices are performed.

For example, the surface forces, the high surface-to-volume ratio, the frequency of medium change under laminar conditions are all factors that can largely affect the experimental outcome.

For all these reasons, translating procedures from macroscale to microfluidic technology is not always possible and feasible.

WO 2013/059343 A1 describes a cell treatment in suspension where cells undergo physical modifications to increase the intracellular delivery of different reprogramming agents, such as proteins, DNA, and RNA.

A second document (Villa M M et al “Culturing primary mouse embryonic fibroblasts in a microfluidic system”, Bioengineering Conference, 2009, IEEE 35th Annual Northeast, IEEE, Piscataway, N.J., USA, 3 Apr. 2009) describes mouse cell culture within a microfluidic device for approximately 7 days.

Another document (Max villa et al: “Growth of primary embryo cells in a microculture system”, Biomedical microdevices, Kluwer Academic Publishers, BO, vol. 12, no. 2, 10 Dec. 2009) describes pluripotent cells seeded into a microfluidic device but produced outside of the device.

According to Yoshinori Yoshida et al (“Hypoxia enhances the generation of induced pluripotent stem cells”, Cell Stem Cell, Elsevier, Cell 20 Press, Amsterdam, NL, vol. 5, no. 3, 1 Sep. 2009), hypoxia is a condition that favors pluripotent cell generation.

### SUMMARY

The present disclosure shows that the processes of cell reprogramming and differentiation can be translated into and integrated within microfluidic technology.

According to a first aspect, a method for cell reprogramming and differentiation by microfluidic technology is disclosed. In particular, an in vitro method for cell reprogramming that includes the step of converting differentiated cells, cultured in adhesion within a microfluidic device, to cells at an embryonic stage is disclosed.

According to a second aspect, a kit useful to implement the methodology of the present disclosure is described. In particular, a kit for cell reprogramming including a microfluidic device including a cell preparation of differentiated cells or a preparation of iPSc cells is described, wherein said cell preparation is adherent to a surface of the microfluidic device.

### BRIEF DESCRIPTION OF THE DRAWINGS

FIG. 1 shows a schematic example of a microfluidic device for cell reprogramming and differentiation.

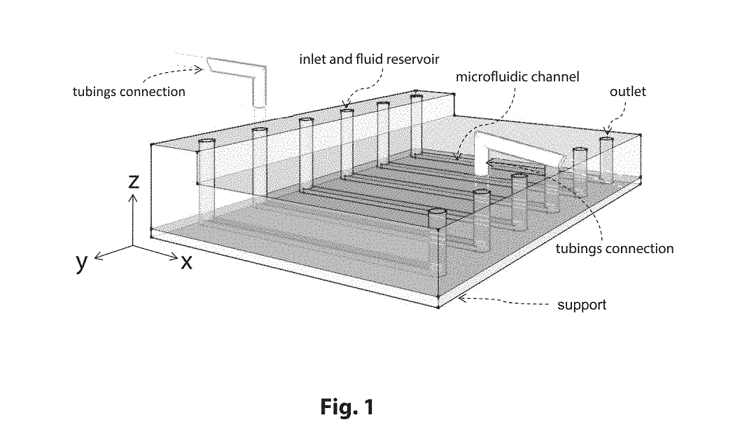

FIG. 2 shows images from bright field microscopy showing an area of a microfluidic channel where BJ cells were cultured for 20 days on different substrates obtained by physical or chemical covalent modification: fibronectin (Fn), gelatin 0.1% (Gel 0.1%), aminopropyl-tri-ethoxy-silane (APTES), APTES with gelatin 0.1%, trimethoxy-silyl-propyl-methacrylate (TMSPMA) and methacrylated gelatin (Gel MA).

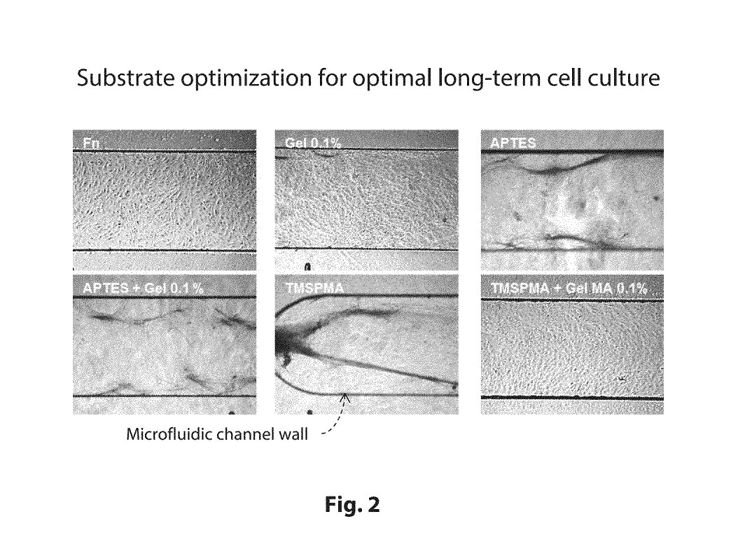

FIG. 3 shows images from fluorescence microscopy showing an area of a microfluidic channel where BJ cells were transfected with mRNA of nuclear EGFP (enhanced green fluorescent protein): bright field (a), nuclei stained with HOECHST 33342 (b), fluorescence of nuclear EGFP (c). Effect of different EGFP mRNA concentrations, delivered on BJ cells in microfluidics for 4 h, on the percentage of transfected cells, detected by EGFP expression after 24 h; results of the transfection performed in a conventional 6-well plate are also presented for reference (d). Effect of different EGFP mRNA transfection durations on BJ cells in microfluidics, at the two indicated concentrations of mRNA.

FIG. 4 shows iPSc colonies generated and expanding within microfluidic channels; a) image of a single channel where iPSc colonies were stained with alkaline phosphatase at the end of reprogramming process performed within the channel; b) formation and expansion of an iPSc colony in the microfluidic channel from day 16 (D16) of the reprogramming process to day 19 (D19). c) Further example of an iPSc colony produced within a microfluidic channel.

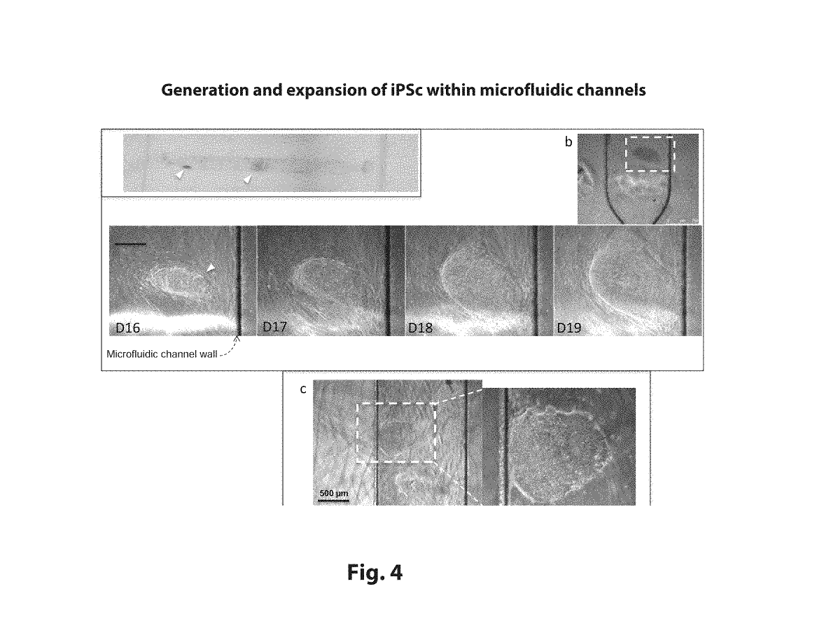

FIG. 5 shows pluripotency markers expression of an iPSc colony produced within a microfluidic channel. Bright field images of the colony before (live) and after fixation by paraformaldehyde. Before fixation, the colony was tested for alkaline phosphatase expression (AP-live) by a fluorescence probe activated by the enzyme. After fixation, immunofluorescence assay was performed for pluripotency markers OCT3/4 and NANOG. Cell nuclei were detected by HOECHST 33342 staining.

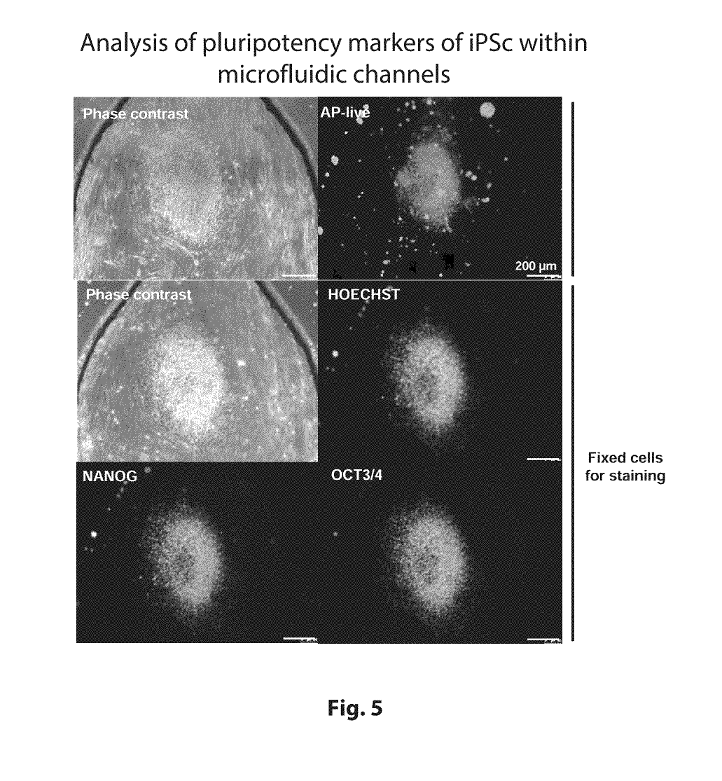

FIG. 6 shows RT-PCR analysis, where total RNA from iPSc produced in microfluidics was extracted and retro-transcribed (RT) to DNA to verify pluripotency markers expression by PCR. iPSc colonies produced in microfluidics are positive for NANOG, OCT4, and SOX2, similarly to iPSc colonies conventionally produced in well and expanded.

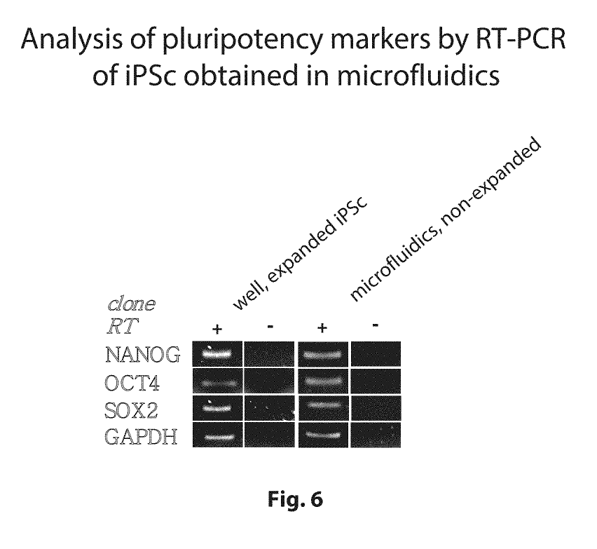

FIG. 7 shows analysis of iPSc pluripotency markers after expansion. iPSc produced in microfluidics were extracted from the microfluidic device and expanded conventionally in well before the analysis (B). Results were compared to those obtained from iPSc produced and expanded conventionally in well (A). In both cases, iPSc maintain pluripotency characteristics after expansion, as demonstrated by immunofluorescence assay of pluripotency markers OCT3/4, NANOG, TRA-1-60, SSEA-4 and the colorimetric assay for alkaline phosphatase (AP). Nuclei were stained with HOECHST33342.

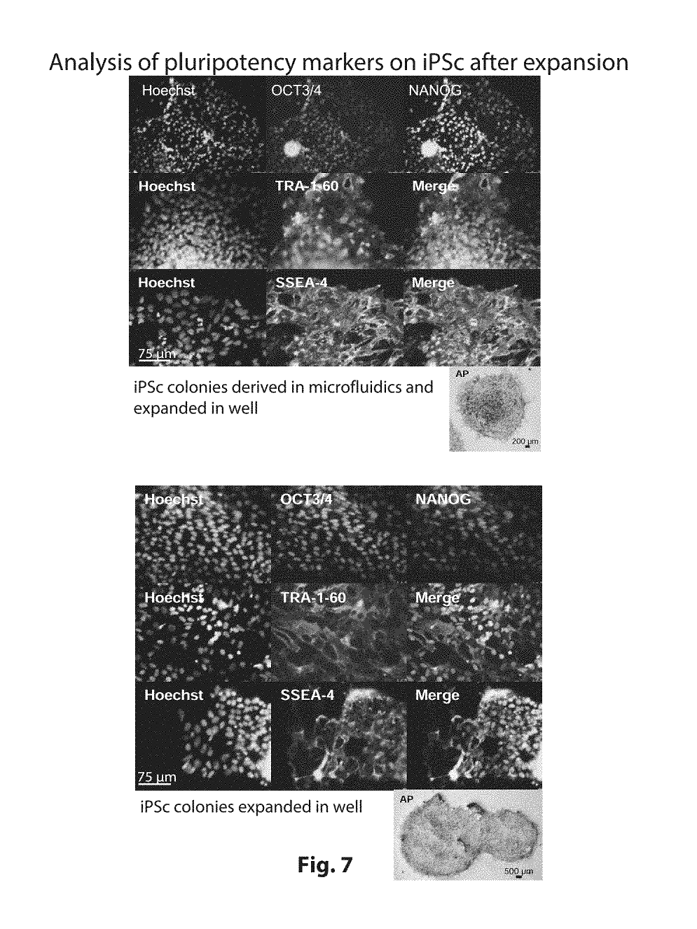

FIG. 8 shows methods for iPSc colony extraction from the microfluidic device: a) rubber coring; b) shear-induced detachment.

FIG. 9 shows aspecific differentiation of iPSc produced in microfluidics without intermediate cell passaging: bright field image of a single iPSc colony during differentiation and RT-PCR analysis to detect expression of markers from the three germ layers.

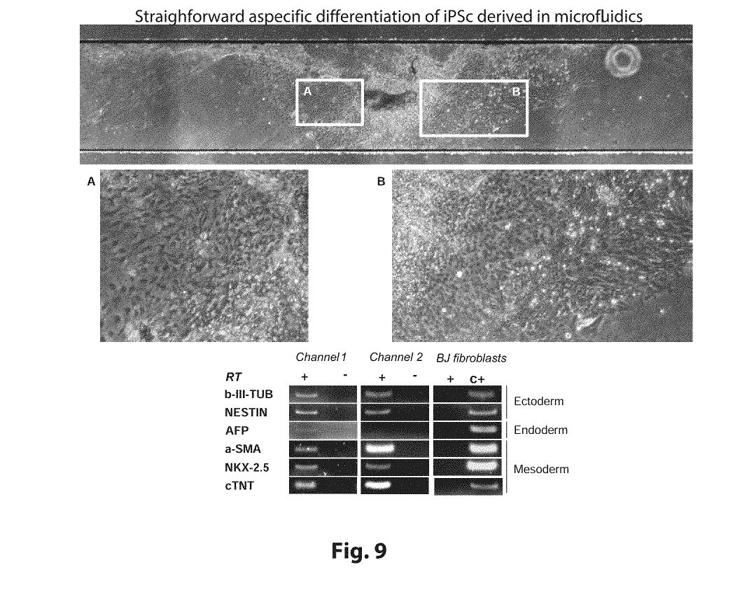

FIG. 10 shows cardiac functional differentiation of iPSc produced in microfluidics without intermediate cell passaging: bright field image of a single differentiating colony and detection of cardiac markers at the end of the protocol by RT-PCR.

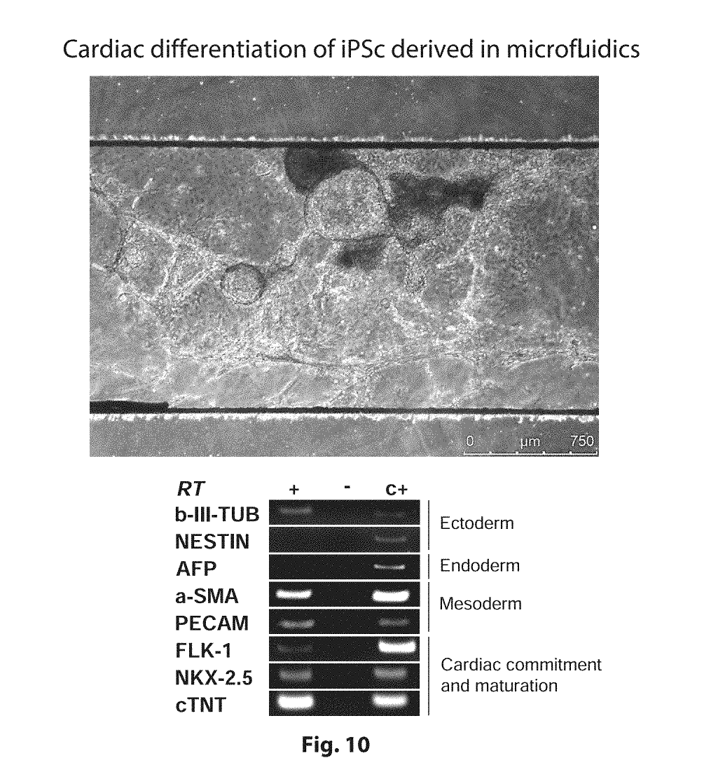

### DETAILED DESCRIPTION

The term “cell reprogramming” herein indicates the process used to de-differentiate differentiated cells towards an embryonic status, known as induced pluripotent cells (iPSc).

The term “embryonic status” herein indicates the status where cells are pluripotent stem cells.

For the purposes of the present disclosure, the differentiated cells to be reprogrammed are preferably adult or neonatal somatic cells, including cells derived from both healthy patients and patients affected by or predisposed to pathologies, for example cancer, including both human and animal cells and, specifically, stabilized and immortalized cell lines or primary cells.

In a preferred aspect of the disclosure, the differentiated cells to be reprogrammed are primary cells, such as human fibroblasts derived from a biopsy, epithelial cells derived from urine samples, and nucleated cells from peripheral and cord blood.

This disclosure also describes a method for performing “cell differentiation”, meaning that undifferentiated cells and, specifically, iPSc are preferentially differentiated towards a defined phenotype.

Preferably, cells undergo an aspecific differentiation process and generate cells belonging to all three germ layers.

Alternatively, cells undergo a specific differentiation process, and generate a cell population enriched in defined cell types.

As described above, for the purposes of the present disclosure, microfluidic devices (microfluidic chips) are used that are currently known.

For example, useful microfluidic chips can have a bottom surface made of glass and a molded circuit made of rubber, such as polydimethylsiloxane (PDMS), assembled after surfaces activation by oxygen plasma, and are sterilizable, for example, by autoclaving.

Specifically, these microfluidic devices are produced by known techniques, for example the known photo-lithography and soft-lithography, and have geometries suitable for the specific application.

For example, they can include one or more culture chambers independent from each other, each, and preferably every, connected with inlet and outlet microfluidic channels.

The microfluidic device can be transparent to allow optical inspection to be performed online during the process, for example when the device is made by a glass bottom and a molded circuit in silicone-like material.

The microfluidic device can have a closed configuration, when inlets and outlets are directly or indirectly connected to sealed reservoirs, making the (closed) system protected from various type of contaminations at device inlets and outlets.

In a preferred aspect of this disclosure, the microfluidic device has a closed configuration.

As the surface of the microfluidic chamber represents the culture surface, it must provide cell adhesion for the whole duration of reprogramming and differentiation (typically 4-8 weeks) and thus it will be made of a suitable material, such as treated glass, polydimethylsiloxane or polystyrene, for example, and can be coated with substrates, by physical adsorption or by covalent chemical bonding, like gelatin (Gel 0.1%), fibronectin (Fn), aminopropyl-tri-ethoxy-silane (APTES), APTES with 0.1% gelatin, trimethoxy-silyl-propyl-methacrylate (TMSPMA), TMSPMA with methacrylated gelatin (Gel MA), etc, or hydrogels (for example derived from polyacrylamide, hyaluronic acid, polylactic acid, polyacrylic acid, etc) or other polymers or biopolymers or inorganic or organic molecules.

In particular, preferred substrates are fibronectin and TMSPMA with Gel MA.

The method for cell reprogramming according to the present disclosure includes in particular the stage of obtaining cells with an embryonic status starting from differentiated cells cultured in a microfluidic device.

Specifically, cells are seeded within the chambers of the microfluidic device at a cell density and under conditions that are dependent on the specific starting cell type.

Moreover, together with these cells, other replication-incompetent cells can be seeded in the device to sustain the process (the so-called feeder layer), for example replication-incompetent human neonatal fibroblasts (NUFF) and replication-incompetent mouse embryonic fibroblasts (MEF), etc.

Reprogramming is performed by the direct or indirect induction of specific factors of intracellular regulation, for example OCT4, NANOG, SOX2, KLF4, MYC, LIN28.

Possibly, for this purpose, cells can also be treated with molecules that directly or indirectly act as epigenetic modifiers, such as valproic acid and sodium butyrate, some hormones or cytokines, organic or inorganic chemical compounds etc, for a suitable period, with the aim of increasing the efficiency of reprogramming.

The techniques that can be used for the purposes of the present disclosure to obtain the reprogramming process can include the use of vectors.

Vectors that can be used to induce gene expression in the cells of the experiment are integrating or non-integrating vectors into the DNA of the host cell, where the term “integrating” indicates the vector that following its integration with the DNA of the host cell replicates itself together with it and is transferred to both the daughter cells at each cell division, whilst the term “non-integrating” indicates the vector that does not affect cell DNA genetic integrity of the host cell.

Non-integrating vectors are distributed between the two daughter cells after each cell division and, thus, after a certain number of cell divisions are diluted to the point that they are eliminated.

Integrating vectors include:
retroviruses;
lentiviruses;
adeno-associated viruses (AAV);
PiggyBac;
   while some non-integrating vectors are:
adenoviruses;
plasmids;
Sendai viruses;
mRNA or other nucleic acids-based molecules;
proteins;
chemical compounds that alone or in combination with other factors induce reprogramming and that can be metabolized by the cell.

Moreover, non-integrating vectors can be classified into vectors with a short lifespan, i.e. little stable as they persist up to 96 hours before being degraded by the cell, and non-integrating vectors characterized by a longer lifespan (>96 hours).

In a preferred aspect of this disclosure, non-integrating vectors with a short lifespan, such as RNA or proteins, are used, where RNA is even more preferred.

As to the obtainment of iPSc, this is verified by alkaline phosphatase staining, immunofluorescence or RT-PCR assays to identify pluripotency markers such as, for example, NANOG and OCT4, and analyses of differentiation ability into the three germ layers.

Following the reprogramming process, the obtained pluripotent cells (iPSc) can be extracted from the microfluidic system for external uses.

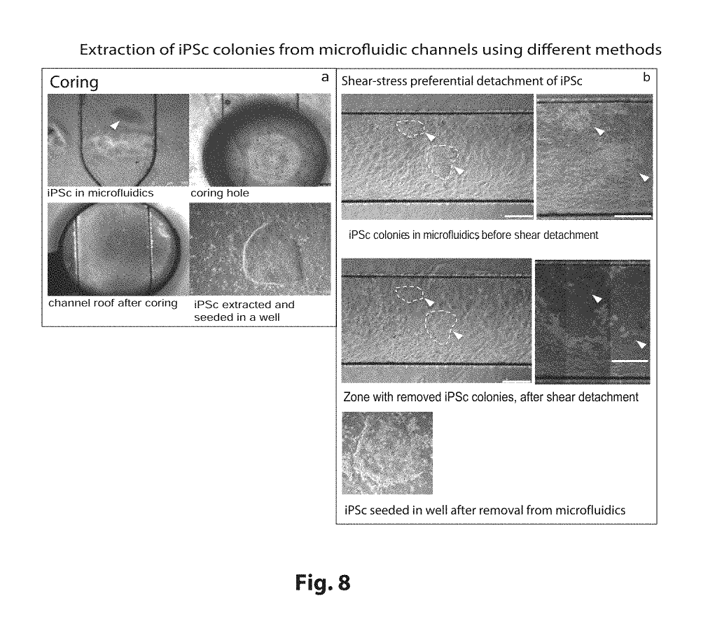

Known techniques for extracting the cells include coring (panel A of FIG. 8), where, due to the possibility of making a hole in the top part of the microfluidic device made of silicon by suitable instruments, the colony can be removed and aspirated.

Alternatively, the iPSc can be detached by flow rates higher than those normally used during culture, which produce a shear stress at the culture surface able to preferentially detach iPSc that can be collected from the outlet flow of the microfluidic device (panel B of FIG. 8)

According to the second aspect of this disclosure, once obtained, pluripotent cells (iPSc) can be differentiated by suitable protocols.

For example, RNAs, cytokines, and culture media containing compounds promoting specific differentiation can be delivered.

During the stages of reprogramming and/or differentiation, hypoxia (hypoxic conditions: <21% of oxygen partial pressure) can be maintained, also not continuously, with the aim of optimizing the efficiency of the processes.

During the stages of reprogramming and/or differentiation, suitable substrates can be used, such as those described above with specific topological, chemical and/or physico-mechanical properties to promote these processes.

In particular, according to the present disclosure, following the reprogramming process and before the differentiation process, an expansion step can be performed.

Such expansion is specifically adopted for getting rid of non-integrating vectors with long lifespan (>96 hours) from the culture.

In a preferred aspect of this disclosure, as reported above, non-integrating vectors with short lifespan are used and the differentiation stage is performed immediately after the reprogramming stage without an intermediate expansion stage.

Even more preferably, the differentiation stage can be performed, and preferably it is performed, within the same microfluidic device where the reprogramming process took place, and also even within the same culture chamber, without previous cell extraction and/or expansion.

Alternatively, reprogrammed cells can be selected and delivered to a different microfluidic chamber of the same or of another microfluidic device, for then proceeding with the differentiation process.

It is also possible to perform, in situ or alternatively outside of the microfluidic device, a purification stage of the iPSc cells separating them from those that were not reprogrammed, before proceeding to the differentiation stage.

On the other hand, in case of use of vectors with high stability in the intracellular environment, like Sendai viruses or plasmids, a prolonged (lasting even weeks) expansion period is necessary.

The occurred cell differentiation is evaluated by verifying cell morphology and expression of specific markers, such as for example β-tubulin, Ncam, Gata4, VEcad, Pecam, Flk1, Gata2, AFP, Nestin, Brachyury.

Following the differentiation stage, the obtained cells, differentiated specifically or aspecifically, can be extracted from the microfluidic device, by techniques known to an average man skilled in the art, or purified.

In a specific aspect, the method of the disclosure is performed in a process following Good Manufacturing Practice (GMP).

According to a further aspect of the disclosure, a kit for performing cell reprogramming and possibly differentiation is described, which includes a microfluidic device, which in turns includes a preparation of differentiated (to be reprogrammed or derived after reprogramming) or pluripotent (iPSc) (produced in microfluidics) cells.

Such cell preparation is adherent to the surface of a microfluidic channel, which can be possibly coated with a suitable substrate or suitably functionalized by physical adsorption or chemical covalent bonding with hydrogels or other polymers or biopolymers or inorganic or organic molecules.

The kit can further include a system integrated with the microfluidic device that allows the performance of one or more stages of the method under hypoxic conditions.

Moreover, the device can be in closed and/or transparent configuration, as described above.

According to a further aspect, the kit of the present disclosure can find application for pharmacological, biological, physiological and pathophysiological analyses.

### Example

A microfluidic chip composed of a glass bottom and a molded circuit of polydimethylsiloxane (PDMS) was assembled by plasma bonding and sterilized by autoclaving.

In particular, the PDMS layer was produced by soft-lithography, with 10 independent culture chambers, each connected to an inlet and an outlet composed by microfluidic channels.

The ends of outlet and inlet channels were mechanically pierced with holes of 1-mm and 3-mm diameter, respectively, throughout the whole thickness of PDMS layer. Large holes at the inlet play as liquid reservoirs.

A microscopy glass slide and the molded circuit of PDMS were treated by oxygen plasma for 2 minutes and brought into contact for 30 minutes at 80° C. for final bonding.

Each microfluidic channel was washed with isopropanol and distilled water to remove processing waste.

The integrated glass-PDMS structure was sterilized by autoclaving for at least 20 minutes at 121° C.

Under laminar hood, a system of needles and sterile tubing was used to connect the outlet holes of the microfluidic channels to a syringe pump that, working in aspiration mode, produces the fluid flow in the culture chambers.

The internal surface of the channels was functionalized by adsorption of fibronectin (0.01 mg/mL) for 30 minutes inside a biological incubator at 37° C. and 5% CO₂.

Human replication-incompetent neonatal fibroblasts (NUFF) were seeded at confluent density in the microfluidic chambers (260 cell/mm²).

After 12 hours, human replication-competent fibroblasts (BJ) were seeded at low density in the same microfluidic chambers (10 cell/mm²).

After another 24 hours and daily for the next 16 days, a freshly-prepared suspension of liposomes and modified mRNA of OCT4, SOX2, KLF4, MYC, NANOG, LIN28 was delivered into the microfluidic chambers for cell transfection by filling the reservoirs at the inlet of the microfluidic chambers.

The suspension was flown into the microfluidic chambers by perfusion at 12 μL/min by the syringe pump and left inside the chambers for 4 hours.

The same pump was used to automatically change the suspension with cell culture medium every 12 hours.

BJ cells underwent a morphological change (˜day 8) and produced iPSc colonies starting from day 10 on, tested by live staining with alkaline phosphatase without affecting cell viability.

During the next days of transfections, new colonies formed while the already present kept on expanding.

The obtained colonies were treated according to the following procedures:
some were analyzed in situ by immunofluorescence (Nanog, Oct4, Sox2, SSEA4, Tra-1-60, Tra-1-81);
others were analyzed by RT-PCR (NANOG, OCT4, SOX2) after RNA extraction;
others were extracted from the microfluidic chamber by means of PDMS layer coring or by perfusion at high flow rate (with shear stress on the cell surface of ˜100 dyne/cm²);
other colonies were aspecifically differentiated directly within the microfluidic chambers by means of a medium composed of Dulbecco's modified Eagles's medium (DMEM) and 10% fetal bovine serum (FBS) and, after 10 days under differentiating conditions, the presence of cells from the three germ layers was verified by RT-PCR (β-tubulin, Ncam, Gata4, VEcad, Pecam, Flk1, Gata2, AFP, Nestin);
other colonies were specifically differentiated towards the cardiac phenotype by inhibitors of Wnt pathway or activators, such as Activin A and BMP4, and, at the end of the protocol the preferential presence of cardiac cells was verified by RT-PCR (c-TnT, Nkx2.5, etc);
the extracted colonies were seeded on inactivated confluent MEFs in standard cell culture dishes and analyzed after expansion for 10 passages by microscopy observation of morphology, immunofluorescence (Nanog, Oct4, SSEA4, Tra-1-60, Tra-1-81) and RT-PCR (NANOG, OCT4, SOX2). Some were used to form embryo bodies (EB) and analyzed by RT-PCR for differentiation markers (β-tubulin, Ncam, Gata4, VEcad, Pecam, Flk1, Gata2, AFP, Nestin).

From the above description, an average man skilled in the art will recognize several evident advantages in the method of the disclosure.

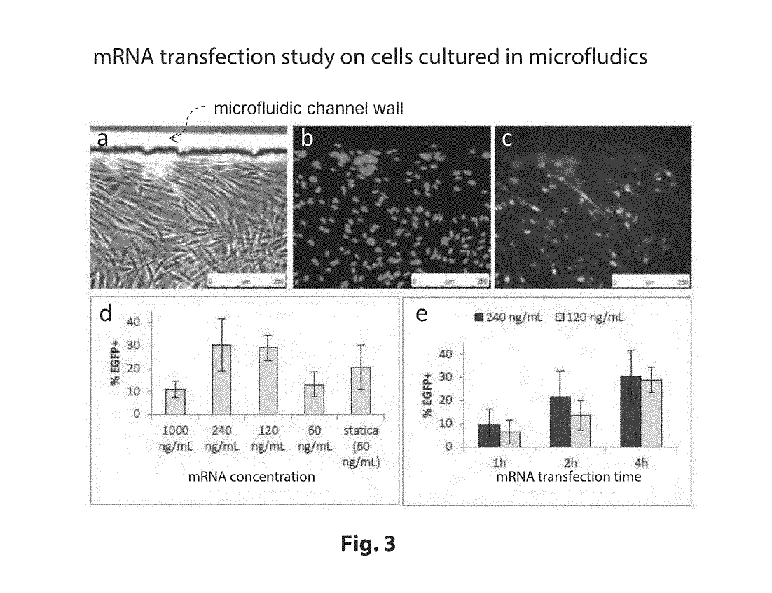

First, using microfluidic technology has some advantages, as it allows a better handling and control of cell microenvironment (concentrations and molecular interactions, for example). In particular, due to the high surface-to-volume ratio, an increase in the efficiency of vector transfections into the cells was verified for the same amount of material used. Even a 4-fold increase of RNA concentration in microfluidics would still use 2.5 folds less amount of material, with respect to protocols performed in conventional dishes according to prior art (FIG. 3).

Moreover, due to the decreased consumption of reagents and the decreased production of by-products, running costs are advantageously reduced.

The significant cost reduction during cell reprogramming stage achievable by performing the process in microfluidics makes economically sustainable performing the subsequent stages of differentiation without need for cell expansion, with a strong reduction in the duration of the whole process, as the iPSc expansion stage usually lasts some weeks.

In the past, this was an important constraint for automating the process because of biological variability of iPSc during the expansion stage.

Moreover, from an environmental point of view, the method of the present disclosure certainly has a decreased environmental impact.

A further advantage of the described method is given by the possibility to use closed microfluidic systems. In this way, operating according to Good Manufacturing Practice (GMP), especially for clinical applications, is feasible.

Moreover, deriving iPSc from cells exposed to pathogens or derived from organisms exposed to pathogens is thus possible.

It is worth noting that the method of the disclosure is prone to be automated, a very interesting aspect from an industrial point of view also because it allows better control and consistency according to GMP rules.

1. An in vitro method for cell reprogramming that includes the step of converting differentiated cells, cultured in adhesion within a microfluidic device, to cells at an embryonic stage.

2. The method of claim 1, wherein said differentiated cells are adult or neonatal somatic cells.

3. The method of claim 1, wherein said differentiated cells are of prenatal origin.

4. The method of claim 1, wherein said differentiated cells are healthy cells or cells derived from patients affected or predisposed to pathologies.

5. The method of claim 1, wherein said cells at an embryonic stage are induced pluripotent stem cells (iPSc cells).

6. The method of claim 1, wherein said cells at an embryonic stage are multipotent or oligopotent.

7. The method of claim 1, wherein said cell reprogramming includes induction of expression of suitable cell regulatory factors into the differentiated cells.

8. The method of claim 7, wherein said induction is performed using integrating or non-integrating vectors into the DNA of a host cell.

9. The method of claim 8, wherein said integrating vectors are selected from the group including retroviruses, lentiviruses, adeno-associated viruses, and PiggyBac.

10. The method of claim 8, wherein said non-integrating vectors are chosen from the group including: adenoviruses, plasmids, RNA-viruses like Sendai, RNA, proteins, and organic or inorganic chemical compounds.

11. The method of claim 7, wherein said induction is performed using non-integrating vectors characterized by a short lifespan.

12. The method of claim 11, wherein said non-integrating vectors characterized by a short lifespan have a lifespan less than or approximately equal to 96 hours.

13. The method of claim 8, wherein said vectors are chosen between RNA and proteins.

14. The method of claim 7, wherein during cell reprogramming the differentiated cells are further treated with direct or indirect epigenetic modifiers, selected from the group including valproic acid, sodium butyrate, hormones, cytokines, and organic or inorganic chemical compounds.

15. The method of claim 1, further comprising, after cell reprogramming, a step of extracting the reprogrammed cells from the microfluidic device.

16. The method of claim 1, further comprising, after cell reprogramming, a step of purifying the reprogrammed cells in situ or outside the microfluidic device.

17. The method of claim 1, further comprising, after cell reprogramming, a cell differentiation step, preceded or not by a purification step.

18. The method of claim 17, wherein said cell differentiation step is performed where the reprogramming step is performed.

19. The method of claim 17, further comprising an expansion step after said cell reprogramming step and before said cell differentiation step.

20. The method of claim 17, further comprising absence of an expansion step after said cell reprogramming step and before said cell differentiation step.

21. The method of claim 17, further comprising, after the cell differentiation step, a purification step in situ or outside the microfluidic device, optionally followed by a step of extraction of the cells from the microfluidic device.

22. The method of claim 1, wherein the step of converting the differentiated cells is performed under hypoxic conditions.

23. The method of claim 1, wherein a surface of the microfluidic device is coated with substrates by functionalization obtained by physical adsorption or by covalent chemical bonding with hydrogels or other polymers or biopolymers or inorganic or organic molecules.

24. The method of claim 2, wherein said somatic cells are fibroblasts of human or animal derivation.

25. The method of claim 2, wherein said somatic cells are replication-competent neonatal or adult fibroblasts.

26. The method of claim 2, wherein said adult somatic cells are of epithelial origin derived from urines, or nucleated cells derived from peripheral or cord blood.

27. The method of claim 2, wherein said adult somatic cells are selected from the group consisting of cells of mesenchymal origin, cells of hematopoietic origin, and cells of amniotic or fetal origin.

28. A kit for cell reprogramming including a microfluidic device including a cell preparation of differentiated cells or a preparation of induced pluripotent stem cells (iPSc cells), wherein said cell preparation is adherent to a surface of the microfluidic device.

29. The kit for cell reprogramming of claim 28, wherein said microfluidic device includes an integrated system for producing hypoxic conditions.

30. The kit for cell reprogramming of claim 28, wherein said microfluidic device includes a surface coated with substrates by functionalization obtained by physical adsorption or by covalent chemical bonding with hydrogels or other polymers or biopolymers or inorganic or organic molecules.

31. The kit for cell reprogramming of claim 28, wherein said microfluidic device is configured as a transparent device.

32. The kit for cell reprogramming of claim 28, wherein said microfluidic device is in a closed configuration.

33. The kit for cell reprogramming of claim 28, wherein said preparation of differentiated cells or iPSc cells is obtained according to an in vitro method for cell reprogramming that includes the step of converting differentiated cells, cultured in adhesion within a microfluidic device, to cells at an embryonic stage.
